(19) United States
(12) Patent Application Publication
(10) Pub. No.: US 2010/0047290 A1
(43) Pub. Date: Feb. 25, 2010

(54) FUNCTIONAL PROTEIN CRYSTALS CONTAINING A CORE NANO-PARTICLE AND USES THEREOF

(75) Inventors: Walther Schwarzacher, Bristol (GB); Oksana Kasyutich, Bristol (GB)

Correspondence Address: Weaver Austin Villeneuve & Sampson LLP, P.O. BOX 70250, OAKLAND, CA, 94612-0250, US

(73) Assignee: UNIVERSITY OF BRISTOL, Bristol (GB)

(21) Appl. No.: 12/593,042

(22) PCT Filed: Mar. 31, 2008
(86) PCT No.: PCT/GB08/01113

§ 371 Date: Nov. 6, 2009

(30) Foreign Application Priority Data
Mar. 29, 2007 (GB) 0706217.7

(51) Int. Cl. A61K 9/00; C07K 14/435; C07K 1/00; C07K 14/195; C07K 14/005

(52) U.S. Cl. 424/400; 530/350; 530/402; 530/400; 977/773; 977/778; 977/906; 977/943; 977/954

(57) ABSTRACT

A functional protein crystal, wherein each protein in the crystal comprises a cavity containing a core nano-particle, the core nano-particle formed from an elemental metal, a metal alloy, or a metal compound, with the proviso that the protein is not apoferritin Dpr or E. Coli dps when the core particle is ferrihydrite.

### FIELD OF THE INVENTION

The present invention relates to a functional protein crystal in which the proteins in the crystal comprise a cavity containing a core nano-particle formed from an elemental metal, a metal alloy or a metal compound. The invention also relates to methods of making such protein crystals. The crystallised proteins, comprising the core-nanoparticles find a variety of uses as discussed herein.

J. Biol. Chem. Vol 277, 2002, Ilari, A. et al refers to the preparation of a crystal of E. Coli dps protein containing ferrihydrite core nanoparticles. The crystal referred to in this reference is not within the scope of the present invention.

J. Mol. Biol. Vol. 364, 2006, Kauko, A. et al refers to the preparation of a crystal of Dpr protein containing ferrihydrite core nanoparticles. The crystal referred to in this reference is not within the scope of the present invention.

### BACKGROUND OF THE INVENTION

3D nanoparticulate arrays are presently generating interest in the fields of optics, electronics and optoelectronics due to their potential applications in areas such as biological sensing, waveguides, data storage and high-resolution imaging.

The usefulness of these arrays is derived in part from the optical and electronic properties of the nanoparticles comprising the array. These properties in turn are dependent on the nanoparticle size and inter-particle spacing and so it is desirable to be able to control these parameters. Lithography has been used to divide a thin film into individual particles of the desired shape. However, this is expensive and is restricted to 2-dimensional arrays. Alternatively, colloidal semiconductor and metal nanocrystals have been developed (Murray, C. B. et al; Annu. Rev. Mater. Sci. 2000. 30, pp 545-610; Synthesis and Characterisation of Monodisperse Nanocrystals and Close-Packed Nanocrystal Assemblies). However, the size of the crystals obtainable using this method is limited and for some applications, it is desirable to employ larger crystal sizes. In addition, the synthesis step associated with the formation of colloidal crystals is comparatively slow. In addition, colloidal crystals suffer from the disadvantage that the optimisation of two important parameters, namely the functionality of the nanoparticles and their ability to self assemble, needs to be done simultaneously. This often represents a significant technological challenge.

Accordingly there is a need for alternative methods to provide 3-D nanoparticle arrays in which the nanoparticle size and interparticle spacing may be controlled, which do not suffer from the above disadvantages.

### SUMMARY OF THE INVENTION

In accordance with a first embodiment of the invention, there is provided a functional protein crystal, wherein each protein in the crystal comprises a cavity containing a core nano-particle, the core nano-particle formed from an elemental metal, a metal alloy, or a metal compound, with the proviso that the protein is not apoferritin, Dpr or E. Coli dps when the core particle is ferrihydrite.

In accordance with a second embodiment of the invention, there is provided a method of synthesising a functional protein crystal as specified in the first embodiment, the method comprising the steps of:
(i) preparing a protein solution, each protein having a cavity capable of accommodating a core nanoparticle;
(ii) synthesising a core nanoparticle within the cavity of at least some of the proteins in the solution, the core nanoparticle formed from an elemental metal, a metal alloy, or a metal compound;
(iii) separating monomeric proteins, each comprising a core nano-particle, from the solution;
(iv) subjecting the monomeric proteins to crystallisation conditions to form a functional protein crystal.

In accordance with a third embodiment of the invention, there is provided a magnetic data storage medium comprising the functional protein crystal of the first embodiment.

In accordance with a fourth embodiment of the invention, there is provided an optical waveguide comprising the functional protein crystal of the first embodiment.

In accordance with a fifth embodiment of the invention, there is provided a sub-diffraction limited lens comprising the functional protein crystal of the first embodiment.

In accordance with a sixth embodiment of the invention, there is provided an enhancement agent for Raman spectroscopy comprising the functional protein crystal of the first embodiment.

In accordance with a seventh embodiment of the invention, there is provided a photonic band gap device comprising the functional protein crystal of the first embodiment.

In accordance with an eighth embodiment of the invention, there is provided a biocompatible implant comprising the functional protein crystal of the first embodiment.

In accordance with a ninth embodiment of the invention, there is provided a surface plasmon resonance biosensor, comprising the functional protein crystal of the first embodiment.

In accordance with a tenth embodiment of the invention, there is provided a drug and/or thermotherapy delivery system comprising the functional protein crystal of the first embodiment.

### FIGURES

FIG. 1 shows an optical image of magnetoferritin crystals in triangular and pyramidal form (crystals (a) and (b) respectively).

FIG. 2 shows an optical image of larger magnetoferritin crystals.

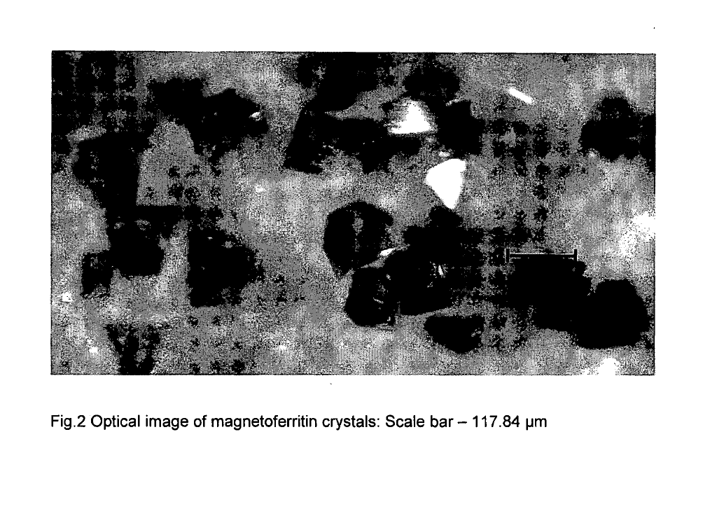

FIG. 3 shows an SEM image of magnetoferritin crystals.

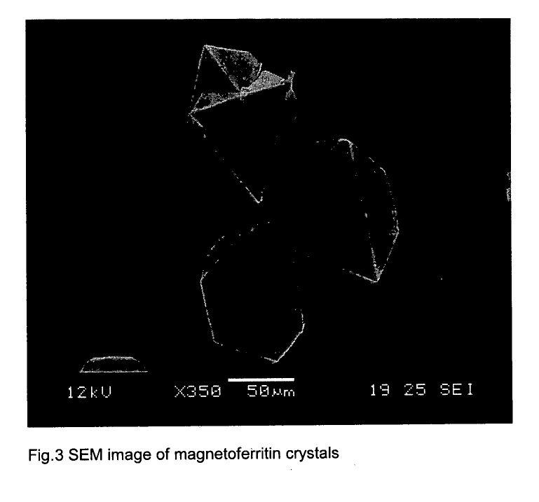

### DETAILED DESCRIPTION OF THE INVENTION

### Protein

The protein for use in the second embodiment of the invention in the preparation of the functional protein crystal of the first and third to tenth embodiments may be any protein capable of crystallisation, which contains a cavity within which a core nanoparticle may be formed and which will at least partially surround the formed core nanoparticle. The protein may be an assembly of protein molecule sub-units, which combine to give a protein with the above properties. The cavity may be almost fully enclosed within the protein with a small channel or channels providing access to the cavity from the external environment in order to allow core material to enter the cavity during synthesis of the core nanoparticle. Alternatively, the cavity may include a larger opening or openings to give a cavity which is not fully surrounded, but which nevertheless is capable of receiving and supporting the core nanoparticle; for example, the opening may be that defined by an annulus in the protein.

The protein may for example be selected from a member of the ferritin family, capsids, viruses (for example the tobacco mosaic virus), bacteriophages, lysozymes (e.g. hen egg white lysozyme), flagellar LP rings, microtubules and chaperonins. The protein may for example be selected from DPS or apoferritin, such as apoferritin e.g human or horse apoferritin. In addition, the protein may be selected from a recombinant or genetically engineered protein.

Natural ferritin has a molecular weight of 900 kD and is utilised in iron metabolism throughout living species and its structure is highly conserved among them. It consists of 24 subunits which self-assemble to provide a hollow shell roughly 12 nm in outer diameter. It has an 8 nm diameter cavity which normally stores 4500 iron(III) atoms in the form of paramagnetic or antiferrimagnetic ferrihydrite. The stoichiometric formula of ferrihydrite is most commonly taken as 5Fe₂O₃·9H₂O but it may also be taken as Fe₅HO₈·4H₂O, Fe₆(O₄H₃)₃, Fe₂O₃·2FeOOH₂.6H₂O and Fe_{4.5}(O,OH,H₂O)₁₂ depending on the water content of the mineral. However, this ferrihydrite can be removed to leave a ferritin unit which is devoid of ferrihydrite and which is termed “apoferritin”. The subunits in ferritin pack tightly; there are, however, channels into the cavity at the 3-fold and 4-fold axes. The presently preferred protein for use in the invention is the apoferritin protein, which has a cavity of the order of 8 nm in diameter. The core nano-particle to be accommodated within this protein will have a diameter up to about 10 nm in diameter, as the protein can stretch to accommodate a larger particle than one 8 nm in diameter.

Ferritin can be found naturally in vertebrates, invertebrates, plants, fungi, yeasts and bacteria. It can also be produced synthetically through recombinant techniques. Such synthetic forms may be identical to the natural forms, although it is also possible to synthesise mutant forms, which will still retain the essential characteristic of being able to act as a template for the formation of nanoparticles and accommodate a nanoparticle within its internal cavity. The protein may, for example, be provided with a modified pattern of surface charged groups in order to induce it to crystallize into a different crystalline form from that of the native protein. The use of all such natural and synthetic forms of ferritin is contemplated within the present invention.

DPS is a ferritin homologue, dodecamer DNA protection protein comprising a hollow core and pores in the three-fold axis.

Flagellar LP rings are ring-shaped structures having an inner diameter of approximately 13 nm and outer diameter of approximately 20 nm.

Microtubules are tubular proteins, formed from αβ-tubulins, and have an outer diameter of about 25 nm and a length of several micrometres.

Chaperonins are protein complexes that assist protein folding. Chaperonins have a structure comprising two stacked rings, each ring having either 7, 8 or 9 subunits, to giving a cylindrical cavity.

### Protein Solution Preparation

In cases where the protein cavity naturally includes core material, for example ferrihydrite in the case of the ferritin protein, this core material must be removed prior to synthesis of the core nanoparticle of the invention in the cavity.

Removal of the native core material may take place by any method known in the art, for example by dialysis against a solution in which the concentration of natural core solutes is maintained at a low level. This process preferably takes place under an inert atmosphere, for example under nitrogen.

In the case of the protein ferritin, the ferrihydrite core may be removed (to give apoferritin) by dialysis against thioglycolic acid in a sodium acetate buffer at pH 4.5, followed by repeated dialysis against 0.15M saline as specified in the example of U.S. Pat. No. 5,491,219 (Mann et al).

### Synthesis of Core Nanoparticles

The core nanoparticles may be prepared by a process in which a solution of the protein material, typically in an aqueous medium, is combined with a source of ions of the appropriate metal, metals or non-metals to comprise or consist the core nanoparticle. The source(s) of ions are preferably added incrementally to the protein solution. For example the cation and anion sources may be added in sufficient amounts to provide more than 1 atom of the cation and anion sources per protein per iteration, preferably more than 20 atoms of the cation and anion sources per protein per iteration. The cation and anion sources may be added in sufficient amounts to provide fewer than 200 atoms of the cation and anion sources per protein per iteration, preferably fewer than 100 atoms of the cation and anion sources per protein per iteration. In a preferred embodiment of the invention the cation and anion sources may be added in sufficient amounts to provide about 50 atoms of the cation and anion sources per protein per iteration. These low concentrations may be achieved by successive dilutions of solutions containing the cation and anion sources.

In aspects of the invention in which the core particle is a metal, it is preferred that the source of metal ions be a salt of the metal or metals, for example an ammonium salt e.g. ammonium tetrachloroplatinate.

Alternatively, but presently less preferred, the source of ions may be present in a composition to which a source of protein is added.

The mixture of protein and ions may be agitated to ensure homogenisation. Where the core nanoparticle is to comprise an elemental metal, a reduction is effected on the composition whereby a nanoscale metal particle forms within the protein cavity.

Where the core nanoparticles are formed by a reductive process, the reduction preferably takes place under an inert atmosphere to prevent aerial oxidation.

Where the core particle is a metal oxide, it may be formed by adding the atoms in a soluble oxidation state and then applying a controlled oxidation to oxidise the metal ions into an insoluble higher oxidation state using an oxidising agent added under an inert atmosphere. For example, where the particle is a γ-Fe₂O₃/Fe₃O₄ core in Magnetoferritin, the iron atoms may be added in a suitable bivalent state and then oxidised into an insoluble trivalent state using an oxidising agent added under an inert atmosphere.

Various chemical oxidants can be used for the oxidation step, including, metal iodate salts, and trimethylamine oxide. Alternatively electrochemical oxidation may be employed.

The reduction/oxidation step may be repeated between additions of core particle source ions (which may be the same or different in each cycle) to build up the nanoparticles. In another aspect, incubation followed by reduction, or continuous additions of the active element and the oxidising agent over a short time (e.g. 5-10 minutes) may be used.

The reaction mixture may be formed at a temperature below the preferred temperature at which the core nanoparticles are allowed to form and then raised to that temperature. Alternatively, the source of protein material to which the source(s) of metal ions is to be added may be held at a suitable formation temperature and the metal ion source(s) added thereto.

The suitable formation temperature will depend on the type of the core nanoparticle. For ease of synthesis, some core particles may be synthesised within the protein in a solution held at room temperature. However, in the case of semi-conducting particles, with the exception of cadmium sulphide, these particles often require a temperature of at least 24° C. in order to form within the protein core.

Proteins can generally withstand temperatures of up to 70° C. before they lose their tertiary structure. Thus the temperature of the reaction may range up to about 70° C. The reaction temperature is preferably maintained in the range from about 25° C. to about 60° C., more preferably in the range from about 35° C. to about 50° C. In some cases, proteins can maintain their thermal stability at higher temperatures. For example, DPS proteins have been reported as being stable up to about 92° C. For such proteins, the reaction temperature may be higher, for example greater than 70° C., such as greater than 80° C. The reaction temperature will be usually be less than 90° C.

In one embodiment of the invention, the aqueous medium is maintained at alkaline pH during the formation of the core nanoparticles within the macromolecular templates. The pH is preferably maintained in the range from 7.5-8.5. This may be achieved by the use of a buffer solution or the use of dynamic titration. Suitable solutions will vary depending on the protein used.

The protein solution may be regarded as a “solution” in the sense that the components thereof are generally regarded as being solubilized, although such solutions can also be regarded as colloidal suspensions. The predominant component of the solution is preferably water, although a percentage of one or more water-miscible solvents may also be present. These water miscible solvents may be present in a total amount of up to 50% by weight based on the weight of the solution. The percentage of water-miscible solvents in the liquid medium is preferably less than 25% by weight, more preferably less than 10% by weight.

The reaction mixture is maintained at the reaction temperature for a time sufficient to permit nanoparticle formation. This may be a time of between 15 and 120 minutes, preferably about 60 minutes.

### Core Nanoparticles

Normally, the core nanoparticles of the invention will have all of their dimensions in the nano size range, typically at least 1 nm and no greater than 100 nm. However, in some aspects, all of the dimensions of the core nanoparticles may be greater than 100 nm, for example greater than 300 nm or greater than 400 nm. In these aspects, the core nanoparticles of the invention will normally have all of their dimensions less than 1000 nm, for example less than 800 nm or less than 600 nm. In an aspect, core nanoparticles of the invention have a diameter (or largest dimension in the case of non-spherical particles) of at least 1 nm, for example at least 3 nm such as at least 6 nm or at least 10 nm. These particles normally have a diameter (or largest dimension in the case of non-spherical particles) no greater than 100 nm, for example no greater than 50 nm, such as no greater than 20 nm or 15 nm. The core nanoparticles of the invention are preferably spheroidal. However, the present invention also extends to core nanoparticles which have one dimension which is not within the nanosize range, as for example, the particles formed using microtubules which are tubular proteins, formed from α,β-tubulins, and have an outer diameter of about 25 nm and a length of several micrometres.

Since the cavity size for a given type of protein is the same as that in other proteins of that type, synthesis of a core nanoparticle within the cavities of proteins of a given type can provide a narrow core nanoparticle size distribution. Typically, the standard deviation in core particle size (diameter in the case of spherical particles and largest dimension in the case of non-spherical particles) is less than 50%, for example less than 30% of the average value. In preferred embodiments, the standard deviation is less than 10%, for example less than 5% of the average value.

In aspects where the core nanoparticle is an elemental metal, the metal may be a transition metal. For example, the elemental metal may be selected from iron, cobalt, nickel, copper, palladium, platinum, silver or gold; for example platinum, silver or gold; or silver or gold.

In aspects where the core nanoparticle is a metal alloy, the metal alloy is a combination of two or more elements, at least one of which is a metal, to give a material having metallic properties, such as conductivity, at room temperature. The metal alloy may contain one or more non-metals e.g. S, P, C, Si, or O. The weight percentage of non-metal in the alloy may be less than 10% or less than 5%, for example less than 3 wt %, or less than 1 wt %. Alternatively, the metal alloy may contain only metal elements.

In aspects where the core nanoparticle is a metal compound, the metal compound comprises at least one metal and one non-metal or semi-metal. The metal compound exhibits either semi-conducting or insulating properties at room temperature, for example semi-conducting properties. In different aspects, the metal compound may be crystalline or non-crystalline, for example crystalline. The metal compound may also be a semi-conductor, a ferro- or ferri-magnetic material or a ferro- or ferri-magnetisable material.

By “ferr- or ferro-magnetisable material”, we mean that the material is either ferr- or ferro-magnetic at room temperature or that it is convertible to such a ferr- or ferro-magnetic form by a structural modification that does not significantly alter the stoichiometric formula of the core material or its mass, for example annealing.

In addition, the metal compound may be a compound exhibiting superconducting properties at a temperature below room temperature.

In some embodiments, the metal element or elements in the compound may include iron. In other embodiments, no iron may be present in the metal compound.

The core particle material and size may be selected according to the properties required for a given application in order to allow the core particle to exhibit the required electrical, magnetic properties and/or optical properties.

Depending on the application, it may be desirable for the core particles to exhibit conducting, semi-conducting or insulating electrical properties. The electrical properties exhibited by a given core particle are largely a function of the core particle material. Elemental metals and metal alloys give core particles with conducting properties and metal compounds give core particles with semi-conducting and insulating properties.

Similarly, depending on the application, it may be desirable for the core particle to exhibit ferro-, ferri-, anti-ferromagnetic, superparamagnetic, paramagnetic or diamagnetic magnetic properties. The magnetic properties exhibited by the core particle depend on factors such as core material, core particle size and core particle form (e.g. crystalline form). These factors may be controlled in order to obtain a particle exhibiting the desired properties.

Of the above elemental metals, in an aspect, gold and silver core nanoparticles may preferably be used in optical applications. In another aspect, platinum, palladium and gold may be used in catalytic applications. In a further aspect, iron, cobalt and nickel may be used in magnetic applications.

In an aspect, the metal alloy may be selected to have useful magnetic properties, for example the metal alloy may be selected to be ferr- or ferro-magnetisable. In this aspect, the metal alloy may contain one or more of the following: aluminium, barium, bismuth, cerium, chromium, cobalt, copper, dysprosium, erbium, europium, gadolinium, holmium, iron, lanthanum, lutetium, manganese, molybdenum, neodymium, nickel, niobium, palladium, platinum, praseodymium, promethium, samarium, strontium, terbium, thulium, titanium, vanadium, ytterbium, and yttrium. The metal alloy may comprise a binary alloy or ternary alloy such as cobalt-nickel, iron-platinum, cobalt-palladium, iron-palladium, samarium-cobalt, dysprosium-iron-turbide or neodymium-iron boride, iron-cobalt-platinum, cobalt-nickel-platinum, or cobalt-nickel-chromium. The metal alloy may comprise cobalt or platinum, for example the metal alloy may be an alloy of cobalt and platinum.

Alternatively, the metal alloy may be selected to have useful optical properties. In this aspect, the metal alloy may contain one or more of platinum, silver or gold; for example the alloy may be an alloy of silver and/or gold.

In a further aspect of the invention, the metal alloy may be selected to have specific optical and magnetic properties by, for example, combining one or more of the metals selected for their optical properties (e.g. silver or gold) with one or more of the magnetically useful metals listed above (e.g. cobalt or iron). The metal alloy thus obtained may for example be selected to exhibit paramagnetic, superparamagnetic, ferr- or ferro-magnetic character depending on the application.

Where the metal compound is a semiconductor, the semiconductor material may be selected from gold sulphide, gold selenide, gold telluride, cadmium sulphide, cadmium selenide, cadmium telluride, zinc sulphide, zinc selenide, zinc telluride and mixtures thereof.

Where the core nanoparticle is a ferr- or ferro-magnetisable material, the material may be selected from a ferr- or ferro-magnetisable elemental metal or metal alloy, for example one of the ferr- or ferro-magnetisable elemental metals or metal alloys previously mentioned. The ferr- or ferro-magnetisable material may also be selected from magnetite; maghemite; a compound of formula MeFe₂O₄, where Me is a divalent transition metal selected from Ti, Cr, Mn, Co, Ni, and Cu; or a ferr- or ferro-magnetisable transition metal sulphide, for example Fe₃S₄ or FeS. Separation of monomeric proteins containing core material from protein solution

Following synthesis of the core nanoparticles within the proteins, it is necessary to separate the other products of the core-particle synthesis step from the monomeric proteins containing core nanoparticles before crystallisation can take place. This is because the synthesis of core nanoparticles within the protein leads to additional undesirable products, which would interfere with crystallisation of the monomeric proteins if retained.

Specifically, the reaction product of the synthesis step typically contains one or more of the following products:
(i) Monomeric proteins, fully and partly filled with core material (the desired product);
(ii) Broken down and agglomerated proteins, with core material associated with them (either within the cavity of the protein or on an external surface);
(iii) Particles of core material unassociated with proteins.

The separation of the monomeric proteins containing core nanoparticles may be accomplished using any suitable technique known in the art.

In a preferred embodiment, separation of monomeric proteins containing core material from the product of the synthesis step is achieved by sequentially subjecting the solution containing the synthesised core nanoparticles to ion exchange chromatography followed by gel filtration.

The ion exchange chromatography step produces a fraction consisting mainly of monomeric proteins that are filled or partly filled with core material (i.e. product (i) above).

Gel filtration (size exclusion chromatography) is used as a final “polishing” step to remove any larger aggregates that may have formed. The separation techniques are described in more detail below.

### Ion Exchange Chromatography

Chromatographic methods separate mixtures of substances using two phases, one of which is stationary and the other mobile. Separation of the components in the mixture takes place due to factors such as differences in charge/charge distribution on the component, relative solubility of the components in the mobile phase, or relative strengths of adsorption of the components on the stationary phase.

Whilst any suitable chromatographic method may be used, an exemplary method for use in the present invention has been found to be ion exchange chromatography, which is capable of separating species with very minor differences in surface charge properties, thereby achieving a high level of fractionation.

Ion exchange chromatography relies on the use of an ion exchanger, which is an insoluble matrix to which charged groups have been covalently bound. The charged groups are associated with mobile counter-ions. These counter ions may be reversibly exchanged with other ions of the same charge without altering the matrix. The progress of the analyte through the ion exchanger is therefore retarded due to coulombic interactions between the charges on the analyte and those on the covalently bound oppositely charged groups.

In the case of a protein analyte, proteins may have positively and negatively charged surface groups giving the protein a net positive or a net negative charge. Whether a given protein exhibits a positive or negative charge at a given pH depends on whether the pH of the solution is above or below the protein's isolectric point (i.e. the pH at which the protein exhibits zero net surface charge). The ion exchanger should therefore be selected to give an oppositely charged covalently bound group on the stationary phase of the column to the net surface charge of the protein at the selected pH i.e. where the protein exhibits a net negative charge at a given pH, a positively charged group should be used on the column (cation exchange chromatography) and where the protein exhibits a net positive charge at a given pH, a negatively charged group should be used on the column (anion exchange chromatography).

Separation in Ion Exchange chromatography is achieved by optimising the reversible adsorption of charged solute molecules to immobilised ion exchange groups of opposite charge. Different substances (for example monomeric proteins, non-protein species, broken down proteins etc) have different degrees of interaction with the ion exchanger due to differences in their net surface charges, charges densities and charges distributions over their surface. Those interactions can be controlled by ionic strength and pH of the mobile phase.

This is typically done by achieving a reversable binding (adsorption) of the target fraction (monomeric proteins) to the ion exchanger and allowing other species (contaminants (ii) and (iii) as specified above) to flow through unbound, or to bind with a different binding strength to the ion exchanger.

One generally applicable technique to obtain a phase consisting essentially of monomeric proteins (filled and partly filled with core material) is to apply the step salt gradient elution technique. In this technique, the salt concentration of the mobile phase is increased in stepwise fashion (thereby increasing the ionic strength) and the content of the corresponding elutions measured in order to determine the salt concentration at which the required monomeric protein phase is obtainable.

The initial salt concentration selected will depend on the protein but will typically be greater than 0.02M, for example greater than. 0.05M and will typically be less than 0.4M, for example less than 0.2M.

The step size in salt concentration may be the same or different for sequential elutions and will typically be greater than 0.05M, for example greater than 0.1M between sequential elutions. The step size may also be less than 1M, for example less than 0.5M.

Any suitable salt may be used, for example sodium chloride.

The content and properties of the fractions obtained in the different elutions may be analysed to determine the composition using any conventional technique e.g. UV-Visible spectroscopy (to detect protein presence), gel electorphoresis (to determine the constituent elements of the elution), small angle light scattering (to determine particle size distribution) and Vibrating Sample Magnetometer (VSM) or Superconducting Quantum Interference Device (SQUID) to determine magnetic properties.

The qualitative composition of each elution is characterised by gel electrophoresis. Each elution contains various amounts of the following species: species unbound to the exchanger; monomeric; dimeric; and higher oligomeric proteins; and agglomerated proteins. The required elution is the elution which contains mostly monomeric proteins.

### Gel Filtration (Size Exclusion) Chromatography

Gel filtration is a technique well known in the protein purification/separation art.

It comprises the use of a gel as the stationary phase in combination with a mobile phase. The principle of operation is that the gel contains pores that are passable to smaller particles but impassable to larger particles. Therefore larger particles (which are unable to enter the pores) pass around the gel particles, thereby travelling through the column more quickly.

The gel should be selected to cover a fractionation range, which includes that of the protein to be separated. The gel is therefore preferably selected to provide its optimal resolution over the range 1-1,000 KDaltons (the size range for typical proteins of interest). A suitable gel giving good resolution over this range is Superdex×200 pg.

The buffer selected will depend on the protein. For example in the case of magnetoferritin, a buffer of 0.2 mM sodium acetate (pH=5) may be used.

Following the gel filtration stage, the filled and partly filled monomeric proteins may then be crystallized.

As mentioned previously, an advantage of the present invention is that the use of the protein cavity as a template results in an improved level of uniformity of the core nanoparticles, due both to the cavity size imparting a constraint on the upper size limit that the core particle may attain and due to the regularity in cavity shape between different protein particles of the same type.

The size distribution may be further improved by the use of a magnetic fractionation step in cases where the core nanoparticles exhibit some magnetic properties, for example in cases where the core particles exhibit paramagnetism, superparamagnetism, ferr- or ferromagnetism. The magnetic fractionation step selects proteins according to the degree to which they are filled with magnetic material. This step further narrows the size distribution of the core nanoparticles, since the magnetic properties of the protein containing the core magnetic nanoparticle are dependent to a large extent on the amount of magnetic material in the cavity. Therefore, magnetic fractionation can be used to select proteins with a similar amount of magnetic material contained in them to obtain protein compositions comprising more uniform core magnetic particles.

If used, the magnetic fractionation step is preferably carried out after the ion exchange step and before the gel filtration step. Further details of the magnetic fractionation step are found below.

### Magnetic Fractionation

Magnetic fractionation involves passing the mixture to be separated through a magnetisable stationary phase under gravity or by the exertion of a positive pressure while subjecting the stationary phase to an external magnetic field in order to magnetise it. This has the effect that the particles within the mixture are spatially separated according to their magnetic properties due to their interaction with the stationary phase; thus providing a means of obtaining a concentrated composition of particles having similar magnetic properties.

The proteins having little or no core material will not interact significantly with the stationary phase (and so will pass more quickly through the column), whereas those proteins having at least some core material will exhibit significant magnetic interaction with the stationary phase (and so will pass more slowly through the column).

The stationary phase may comprise steel, for example type IV 20L, or another suitable soft-magnetic material (e.g. Fe—Ni alloy) in the form of a powder, beads, wool or other form known in the art.

One skilled-in-the-art would appreciate that many means for magnetic fractionation are available such as magnetic wire, magnetic powder chromatography and field-flow fractionation techniques.

In the present invention, a preferred technique is High Gradient Magnetic Separation (HGMS). The principle of HGMS is that by creating high localised magnetic field gradients in the stationary phase, magnetic particles passing through this phase will tend to be retained, whereas non-magnetic particles will pass through the stationary phase. This technique has the advantage that more weakly magnetic particles such as those exhibiting paramagnetic character may also be separated from non-magnetic particles.

A typical HGMS arrangement will employ a solenoid electromagnet as the source of the external field, surrounding a magnetic powder (e.g. stainless steel) as the stationary phase.

The flow rate of the protein composition through the stationary phase and the intensity of the external magnetic field applied to the stationary phase are selected according to whether a narrow core particle size distribution or a high yield of proteins containing at least some core material is required.

In cases where the core particle size distribution is less important than a high yield of particles containing at least some magnetic core material, the magnetic field applied to the stationary phase will be selected to be high (for example 0.5-5T), and the flow rate to be low (for example less than 0.3 ml min⁻¹). This will result in the proteins containing a negligible amount of core material passing through the column, whereas those with at least some magnetic core material will be retained. Once the low content proteins have been eluted, the external magnetic field may be removed and the remaining magnetically captured proteins collected.

Conversely, if the applied field is reduced (for example to less than 0.5T) or the flow rate increased (for example to greater than 0.3 ml min⁻¹), a higher proportion of proteins containing a low but non-negligible amount of magnetic material will also be eluted. By adjusting the flow rate or external magnetic field it is therefore possible to adjust the size distribution of the core magnetic nanoparticles.

In a preferred embodiment of the invention, the composition is passed through a column comprising stainless steel powder having a grain size in the range 10-30 μm, for example about 20 μm, at flow rates ranging from 0.05-10 ml/min⁻¹, with an applied external field in the range from 0.1-5T.

### Protein Crystallisation

Any suitable protein crystallisation method may be used in the second embodiment of the invention. In general, the protein may be crystallised by preparing a protein solution and then adjusting the solution conditions in order to obtain a super-saturated protein solution from which protein crystals may be formed.

A supersaturated solution may be obtained in a number of ways, for example by adjusting the pH or temperature of the protein solution; by adjusting the ionic concentration of the solution (for example by adding a crystallizing agent); or by allowing some of the solution to evaporate in order to increase the concentration of protein in the remaining solution.

Where the solubility of the protein is reduced by adjusting the pH, this is done by adjusting the pH of the protein solution to a value closer to its isoelectric point (at which point the protein exhibits its lowest solubility). In the case of ferritin, the isoelectric point is in the pH range of about 4.0-7.0 depending on the ferritin type. Therefore, in the case of proteins in the ferritin family, solubility can be decreased (and therefore crystallisation induced) by adjusting the pH to a value closer to the isoelectic point.

Where the solubility of the protein is reduced by adjusting the temperature, this is typically done by reducing the temperature of the solution. As mentioned previously, protein structure can be damaged by heating to temperatures above about 70° C. Therefore the protein crystallisation process typically comprises the step of dissolving the protein in solution to a concentration at or slightly below the saturation level and at a temperature below 70° C. followed by reducing the temperature in order to induce crystallisation. In cases where the protein is more temperature resistant (e.g. where the protein is DPS), the crystallisation temperature may be higher, for example less than 90° C. The final crystallisation temperature is usually greater than about 0° C., for example greater than about 10° C. or greater than about 20° C. This crystallisation temperature is also usually less than 60° C., for example less than 45° C., such as less than 30° C. Lower crystallisation temperatures, for example less than 30° C., are preferred in order to reduce the possibility of damage to the crystal.

Where the solubility of the protein is reduced by adding a crystallizing agent, this agent may be selected from any commonly used crystallizing agent known in the art. These include salts, high molecular weight straight chain polymers and organic solvents.

Where the crystallizing agent is a salt, the ability of the anion and cation of the salt to precipitate a given protein is given by the Hofmeister series, which may be expressed as follows:

Anions: F⁻, SO₄²⁻>HPO₄²⁻>citrate>acetate>Cl⁻>NO₃>Br⁻>ClO3⁻>I>ClO₄⁻>SCN⁻

### Cations: NH₄⁺>K⁺>Na⁺>Li⁺>Mg²⁺>Ca²⁺

The appropriate salt may therefore be selected from the above anions and cations in order to give a salt with suitable properties to induce crystallisation in the given protein.

The relative proportions of the crystallising agent and the protein determine the nucleation and growth rate of the crystal. Optimisation of these parameters may therefore be used to control crystal size. In addition, further improvement in crystal size may be achieved by suppressing nucleation, for example using the concentration pulse technique.

The concentration of the salt crystallizing agent in the solution will typically be greater than about 0.5 wt % based on the weight of the protein in solution, for example greater than about 1.0 wt %, such as greater than about 2 wt %. The concentration of the salt in the solution will typically be less than about 10 wt % based on the weight of the protein in the solution, for example less than about 7 wt %, such as less than about 5 wt %.

Modification of the salt crystallisation agent also permits control of the crystal form of the protein. For example, in the case of the ferritin protein, the CdSO₄ crystallisation agent has been reported to influence the morphology of ferritin crystals. Apoferritin has been found to form crystal structures such as tetragonal, orthorhombic or cubic. Similarly, hen egg white lysosyme can crystallize in tetragonal, orthorhombic, monoclinic and triclinic forms. Therefore, use of the appropriate crystallizing agent (e.g. NaCl, CdSO₄ or Poly Ethylene Glycol (PEG)) enables the selection of the desired crystalline form of the protein e.g. triclinic, monoclinic, orthorhombic, hexagonal, rhombohedral, tetragonal, or cubic.

In the case of the protein ferritin, sodium chloride, cadmium sulphate and PEG have been reported as suitable crystallizing agents.

Where the crystallizing agent is a high molecular weight straight chain polymer, this polymer may for example be selected from polyethylene glycol, dextran, polyvinyl alcohol, and polyvinyl pyrrolidone, preferably polyethylene glycol.

Where the crystallization agent is an organic solvent, it may for example be selected from straight or branched C₁₋₆ alcohols (e.g. ethanol, methanol, isopropanol, or tert-butanol), acetone or DMSO.

An important factor in the crystallisation of the protein is the protein purity. Impurities lead to defects in the crystal structure, which can lead to poisoning of the growth surface, limiting crystal size and quality. This is particularly significant in the growth of larger crystals, for example those of greater than about 100 μm. The protein crystallisation solution therefore preferably contains less than 0.1 wt %, more preferably less than 0.01 wt % and most preferably less than 0.001 wt % of impurities based on the weight of the protein in solution. By impurities we mean particles capable of reducing the efficacy of the crystallisation process, such as dust particles or other proteins. This level of protein purity is readily achievable using the previously described chromatographic separation processes.

In a preferred aspect of the invention, the batch crystallisation method may be used. In this method, the monomeric protein solution is combined with a buffer and crystallisation agent and then allowed to crystallise. This method has the advantage that it is less susceptible to impurity effects than many other methods, and so produces larger crystals. In addition, the use of a solution in which the components are present under crystallization conditions allows the protein solution to be applied directly to a substrate prior to crystallization in order to permit the formation of crystals e.g. thin film crystals on a substrate. The substrate used will depend on the application but may for example be glass. The film thickness will also depend on the application but may for example be greater than 50 nm, for example greater than 0.1 μm or greater than 0.5 μm, such as greater than 1 μm. The film thickness may also be less than 200 μm, for example less than 100 μm or less than 50 μm, such as less than 10 μm. The crystalline thin film thickness may also be greater than 2 protein units, for example greater than 5 protein units, for example greater than 10 protein units such as greater than 20 protein units. The film thickness may also be less than 200 protein units, for example less than 100 protein units or less than 50 protein units.

Alternatively, the vapour diffusion, hanging drop or sitting drop methods may be used.

In an aspect, the buffer is selected to maintain the pH of the solution at a value close to the isoelectric point, since it has been found that there is a correlation between crystal nucleation pH and pl. For example, the buffer may be selected to maintain the pH within about 3 units of the isoelectric point, preferably within about 2 units, and more preferably within about 1 unit of the isoelectric point.

Therefore in the case of protein in the ferritin family, the solution is preferably buffered to a pH value in the range from 4-7. In the case of horse spleen ferritin, the solution is preferably buffered to a pH in the range from 4-6, for example about 5.

The crystallisation agent in the batch crystallisation method is selected from those listed above. In the case of proteins in the ferritin family, the crystallisation agent may optionally be a metal sulphate, or a metal chloride e.g. sodium chloride.

In accordance with the present invention, the functional protein crystal size (measured in terms of the largest dimension of the crystal) may be greater than 50 nm, for example greater than 0.1 μm, greater than 0.5 μm, greater than 5 μm, greater than 50 μm, greater than 100 μm. In some embodiments of the invention, the functional protein crystal may be greater than 500 μm, such as greater than 1 mm. The functional protein crystal size may also be less than 5 mm, for example less than 1 mm, less than 500 μm or less than 200 μm.

As would be understood by the person skilled in the art, the functional protein crystal may be a 2D or a 3D crystal, for example a 3D crystal.

A 2D crystal comprises protein units arranged periodically to give a crystal that has a nominal thickness of a single protein unit. Where the functional protein crystal is a 2D crystal, the crystal is preferably formed on a substrate, in order to provide a template for crystallisation to take place. However, a template is not essential for all proteins to form 2D crystals. For example, chaperonin proteins may form a 2D crystal in solution without the need for a substrate to be present.

By contrast, a 3D crystal comprises protein units arranged periodically in a three dimensional structure.

In the case of the functional 3D protein crystals of the present invention, each dimension of the crystal when measured through the centre of mass may be equal to or greater than 2 protein units, such as greater than 5 protein units, for example greater than 10 or 50 protein units, or greater than 500 protein units, such as greater than 5,000 protein units or greater than 50,000 protein units.

Each dimension of the crystal when measured through the centre of mass may be equal to or greater than 50 nm, such as greater than 125 nm, for example greater than 250 nm or 1.250 μm, greater than 12.5 μm, or greater than 125 μm, such as greater than 1.25 mm.

Each dimension of the crystal when measured through the centre of mass may be less than 1,000,000 protein units for example less than 500,000 protein units, such as less than 250,000 protein units or less than 100,000 protein units. Each dimension of the crystal when measured through the centre of mass may also be less than 50,000 protein units, for example less than 25,000 protein units, such as less than 10,000 protein units or less than 5,000 protein units.

Each dimension of the crystal when measured through the centre of mass may be less than 25 mm for example less than 12.5 mm, such as less than 6.25 mm or less than 2.5 mm. Each dimension of the crystal when measured through the centre of mass may also be less than 1.25 mm, such as less than 625 μm, for example less than 250 μm or less than 125 μm.

In addition, for example in the case of a 3D crystal crystallised on a substrate, one of the dimensions of the crystal may be less than 100,000 protein units (or less than 2.5 mm) for example less than 50,000 protein units (or less than 1.25 mm), such as less than 25,000 protein units (or less than 625 μm), less than 10000 protein units (or less than 250pm) or less than 5,000 protein units (or less than 125 μm), such as less than 2,000 protein units (or less than 50pm) or less than 1,000 protein units (or less than 25 μm), such as less than 500 protein units (or less than 12.5 μm), or less than 100 protein units (or less than 2.5 μm), such as less than 50 protein units (or less than 1.25 μm) or less than 25 protein units (or less than 0.625 μm), with the remaining dimensions being greater than the specified value.

In this aspect, the shortest dimension may be two or more protein units (or 50 nm or more), or greater than 5 protein units (or greater than 125 nm) such as greater than 10 protein units (or greater than 250 nm).

In another aspect, all dimensions of the 3D crystal when measured through the centre of mass are within a factor of 50 of each other, for example within a factor of 30 of each other, such as within a factor of 10 of each other, or a factor of 5 of each other. In other words, the ratio of the largest dimension to the shortest dimension when measured through the centre of mass for a given crystal of this aspect is within the above range.

The size of the protein crystals of the invention may be measured using any suitable technique, for example optically or by SEM or, in the case of 2D or thin films, by scanning probe microscopy.

The use of a 3D functional protein crystal confers a number of advantages in various technological applications compared to the use of a 2D crystal. For example, in data storage applications, the storage capacity per unit surface area required is greatly increased. The 3D structure of the functional crystal also allows the crystal to be used advantageously in optical applications (e.g. SERS) in which the incident light has at least a component out of the plane of the crystal surface.

In general the use of 3D crystals, in optical devices is advantageous because it allows electromagnetic radiation having a wider range of polarisation states to interact with and propagate through the device in question.

3D crystals also exhibit considerably greater mechanical strength than 2D crystals.

### APPLICATIONS

The crystals of the present invention find applications in a number of fields as detailed below. These applications broadly involve the use of the crystals of the invention as electromagnetic media, optionally having an electromagnetic bandgap, in the propagation and manipulation, including storage, of electromagnetic radiation.

The crystals of the invention may therefore be used as photonic, plasmonic, magnonic and magnetic crystals, composites and metamaterials.

The uses to which the crystals of the invention may be put depend in part on the properties of the core material. For example, in cases where the core material is ferr- or ferro- magnetic, superparamagnetic or ferr- or ferro-magnetisable, the crystal may be used in magnetic or magnonic applications, such as data storage applications or for manipulating magnetic signals as part of a spintronic system.

Alternatively, in other aspects where the core particle are any of metals, metal alloys or metal compounds, the protein crystal may be used in photonic or plasmonic applications such as optical waveguides, sub-diffraction limited lenses, Surface Enhanced Raman Spectroscopy (including single molecule detection), Surface Plasmon Resonance (SPR) biosensors and photonic band gap materials .

### Magnetic Data Storage

In cases where the core nanoparticles are ferro- or ferr- magnetic or ferr- or ferro- magnetisable, the crystal can be used as a component in a 3-dimensional magnetic data storage medium, with each core particle capable of storing a bit of information. Such a medium would provide a significantly higher storage density than the 2-D arrays of magnetic particles (so-called patterned media) that are already being developed in the industry.

In an aspect, the crystal may be grown on a substrate (e.g. glass) prior to use in the data storage medium. In cases where the core nanoparticles are ferro- or ferri-magnetisable but are not ferro- or ferr-magnetic on formation, they may be annealed to make them ferro- or ferri-magnetic. The annealing process may also be used to bind the protein crystal to the substrate more securely.

Reading/writing of the data contained in the core particles in the different layers of the crystal may be accomplished using holographic and magneto-optical techniques. Biocompatible implants, drug and/or thermotherapy delivery systems

The use of a protein crystal in medical applications, for example as an implant or component in an implant, gives the advantage that improved biocompatibility is achieved, particularly where the protein is human-derived (e.g. human apoferritin) and where the core material is iron based (for example magnetite/maghemite).

In addition, the protein is easily functionalised with ligands selected to bind to complementary ligands on other components of the implant in order to improve binding between the crystal and the other component.

The crystal may for example be employed as an optical implant such as a lens or a component in such an implant.

In these aspects of the invention, the protein is preferably human apoferritin with an iron based core, e.g. magnetite/maghemite.

In another aspect, the functional crystal may be used for the targeted delivery of a therapeutic agent or to itself deliver a therapeutic effect (for example by hyperthermia) to a specific location in a patient's body. For example, the functional crystal (which may comprise platinum and/or Au and/or Fe₂O₃ core nanoparticles) may dissociate (either spontaneously or by the use of an external electromagnetic radiation device) to release an active agent (e.g. a drug) at the target location. In the case that the functional crystal is dissociated using external electromagnetic radiation, this radiation may be used to transfer energy to the crystal to produce a local microscopic hyperthermia. In an aspect, the functional crystal may be magnetically guided to or localised at the target location prior to dissociation.

### Optical Waveguides for Nanoscale Devices

Traditional metallic-pipe waveguides suffer from the disadvantage that they are restricted in the EM frequency and degree of curvature of optical path for which they can act as waveguides without significant radiation losses. Specifically, although these traditional waveguides can transmit microwaves along tightly defined paths with negligible losses, transmission of IR and visible light is restricted to straight and moderately curved paths.

Due to the regular ordering of the proteins in the crystal of the invention, in cases where the core nanoparticles are metals or metal alloys, the protein may be selected to provide a geometry of the metal core nanoparticles such that the coupled surface plasmon modes of these core particles propagates EM radiation coherently with negligible radiation losses. Such a waveguide may be used to transmit light along tightly defined paths such as 90° corners and T-shaped structures. In this aspect of the invention, the core metal nanoparticle is preferably silver or gold, for example gold.

### Sub-Diffraction-Limit Resolution Lenses

Classical optical devices are limited in their resolution by the fact that they are only. capable of transmitting the propagating components emanating from a source. Sub-wavelength information is also carried on evanescent waves, which decay exponentially from the surface. However, by contacting the surface with a crystal as specified in the first embodiment of the invention, in which the core nanoparticles are metallic, it is possible to enhance the evanescent field to the extent that sub-wavelength resolution may be attained. In this aspect the core metallic nanoparticles may, for example, be formed from gold, silver or platinum. These metals may be present in elemental form or as alloys. For example, the metal or metals may optionally be combined with a metal selected for its magnetic properties as previously specified, to form an alloy having specific magnetic properties e.g. paramagnetism, superparamagnetism or ferro- or ferri-magnetism. The alloy may for example be selected from gold and a metal selected for its magnetic properties, e.g. cobalt.

### Enhancement Agent for Raman Spectroscopy

Surface electric field intensity due to incident electromagnetic radiation (on which the Raman effect depends) has been shown to be greatly enhanced by the presence of closely spaced metal nanoparticles on the surface [Zou, Shengli et al; Chem. Phys. Let. 403 (2005) 62-67; Silver nanoparticle array structures that produce giant enhancements in electromagnetic fields]. This effect is further enhanced by the alignment of the metal nanoparticles in a periodic array. Therefore, the protein crystal of the present invention in which the core nanoparticles are metal or metal alloys (e.g. silver or gold) may be used as a surface exhibiting an enhanced Raman effect on which samples could be studied.

This could be used for example in high sensitivity applications such as single molecule detection.

In some aspects, the protein may be modified with a ligand complementary to a ligand on the molecule to be detected. Suitable binding elements depend on the application but may for example include an oligonucleotide or monoclonal antibody capable of binding to the target molecule.

In an aspect, the crystal may be grown on a substrate (e.g. glass) as a thin film protein crystal. The glass substrate comprising the protein crystal overlayer may then be used in a standard Raman spectrometer to detect target molecules.

### Surface Plasmon Resonance Biosensors

Surface Plasmon Resonance biosensors are based on the principle that changes in the dielectric constant of a composition of metallic particles give rise to a shift in the Localised Surface Plasmon Resonance (LSPR) spectrum of that composition. This change in dielectric constant may be caused by adsorption of a substance onto the surface of the biosensor.

The functional crystals of the invention, having an elemental metal core particle e.g. gold, silver or platinum may be used as the biosensor substrate on which target molecules are adsorbed.

In some aspects, the protein may be modified with a ligand complementary to a ligand on the molecule to be detected. Suitable binding elements depend on the application but may for example include an oligonucleotide or monoclonal antibody capable of binding to the target molecule.

The LSPR spectrum may be measured using a UV-vis spectrophotometer and so in an aspect the crystal may be prepared on a substrate that transmits light in this range (e.g. UV transmitting glass such as borosilicate glass).

### Photonic Band Gap Devices

Photonic band gap devices comprise materials that are periodic in structure and are organised into photonic bands separated by gaps in which propagating states are forbidden.

Applications of these materials include mirrors, waveguides (see above) and data processing.

### EXAMPLES

The invention is now illustrated by way of the following worked examples.

### Example 1

### Preparation of Magnetoferritin

Apoferritin, i.e. ferritin without its normal iron oxide ferrihydrite core, was prepared from naturally occurring mammalian ferritin using the method specified in Mann et al (U.S. Pat. No. 5,491,219).

Horse spleen ferritin (Boeringer, Cd-free, 50 mg/ml) was dialyzed under nitrogen flow against thioglycolic acid in a sodium acetate buffer at pH 4.5, followed by repeated dialysis against 0.15M saline, to ensure removal of the naturally occurring ferrihydrite iron in the ferritin. A portion of the resulting 1 μM apoferritin solution, buffered to 8.5 pH with 0.05M AMPSO (3-[1,1-dimethyl-2-hydroxyethyl)-2-amino]-2-hydroxypropane sulfonic acid; ex Sigma) was equilibrated in a water bath at 55-60° C. while being purged with argon.

Trimethylamine-N-oxide (Me₃NO) was heated in an oven at 80° C. for 15 mins to remove Me3N. An aqueous solution of Me₃NO (114 mg/ml, 0.07M) and ferrous ammonium sulfate (Fe²⁺; 600 mg/ml, 0.1 M) were prepared and gently deaerated with Ar or N2 for 30 min before use. HEPES buffer solution (0.05M) at pH=8.6 was deaerated for 1 hour, purging with N₂. Apoferritn (100 mg, 0.22 μmol) was added to give a protein concentration of 0.44M. Gentle stirring and deaeration with N2 was continued for another 30 min, after which the reaction vessel was placed in a preheated (65° C.) water bath and the contents allowed to reach equilibrium. Purging with N₂ was continued at a slow rate just above the solution surface, maintaining a positive pressure of N₂. Aliquots of Fe(II) and Me₃NO were added dropwise to the reaction solution using a glass barreled syringes. In general, each addition of Fe(II) was followed by a stoichiometric aliquot of Me₃NO (3Fe(II):2Me₃NO) and the solution left for 15 min before repeating the stepwise procedure. In a typical example, 10 stepwise cycles were undertaken to give a loading of 1000 Fe atoms/molecule (Ref: Biomimetic Synthesis and Characterization of Magnetic proteins(magnetoferritin) , K. Wong, T. Douglas, e.a. Chem Mater., 1998, 10, 279-285).

### Example 2

### Purification of Magnetoferritin

The reaction product of Example 1 was analysed by gel electrophoresis with staining for protein (coomassie blue) and for Fe³⁺ (potassium ferrocyanide in 2.0M HCl), and found to contain the following components:
(i) Magnetoferritin (undisrupted ferritin proteins, fully and partly filled with magnetic core of γ-Fe₂O₃/Fe₃O₄);
(ii) Broken down and agglomerated proteins, with magnetic particles of γ-Fe₂O₃/Fe₃O₄, associated with them; and
(iii) Non protein particles (γ-Fe₂O₃/Fe₃O₄).

The magnetoferritin proteins were isolated from the remainder of the reaction product of Example 1 by the sequential use of (1) ion exchange chromatography, (2) Magnetic separation and (3) Gel filtration (Size exclusion chromatography).

### (1) Ion Exchange Chromatography

Ion exchange chromatography with step salt gradient elution was used to extract the heterogeneous magnetoferritin fraction (fraction (i) above) from the product of Example 1.

### Column Matrix

It is known that the protein ferritin's net surface charge is negative at pH>4 (pl (isoelectric point) for ferritin is 4, pl=4). Therefore a positively charged exchanger (anion exchanger with exchangeable counter-ions) comprising highly cross-linked aragose beads of high chemical and physical stability was used (DEAE Sepharose Fast Flow, diameter 90 μm were used although ANX Sepharose, diameter 36 μm could also be used).

### Choice Of Buffer and Ionic Strength

HEPES buffer (concentration 50 mM) with pH=8 was used as the buffer solution, which is the same buffer as used at the synthesis stage (see Example 1). NaCl ionic strengths for salt-gradient elution were tested and selected to be 0.1 M, 0.25M, 0.5M.

Separation was carried out in a column in the following stages: column equilibration; sample application and absorption; desorption (ion exchange and elution); column regeneration.

Column calibration using apoferritin was primarily performed with the aim of determining elution profile. It was found that the 0.25M NaCl elution consisted of predominantly monomeric proteins with 450 kDa molecular weight (which corresponds to apoferritin), with some quantities of higher oligomeric proteins present (corresponding to apoferritin dimers and trimers). This was tested and confirmed by a quality control procedure, using the gel electrophoresis technique.

### Ion Exchange Procedure

The chromatographic separation process was carried out using the following steps:
1. The column was set up, packed with the medium and soaked with activating buffer over night;
2. The sample was loaded (the product of Example 1, primarily filtered with a 0.1 μm membrane);
3. The column was washed using a starting buffer to exclude all unbound species;
4. Elution took place with 0.1M NaCl;
5. Elution took place with 0.25M NaCl;
6. Elution took place with 0.5M NaCl ;
7. Regeneration of the column was carried out by washing with the starting buffer.

Elutions were monitored by UV/Vis spectrophotometer on the column exit, by measuring specific protein absorbance at 278 nm. Each fraction was collected into individual storage flasks. Qualitative analysis showed that the 0.25 M fraction consisted of unaltered microheterogeneous ferritins. Those proteins are partly or fully filled, and also unfilled. In order to remove the oligomeric and unfilled proteins from the mixture, a further purification step—High Gradient Magnetic Separation—was used.

### (2) Magnetic Fractionation

Magnetic fractionation on a High Gradient Field Flow separator was employed to extract magnetoferritin, filled with an iron oxide core. To generate a high gradient of magnetic field within the volume of the separator, a permanent magnet was used (B˜0.6 T) to saturate the stainless steel powder of the column matrix.

The sample (0.25 M—fraction after ion exchange) was applied to the column while the column matrix was magnetised by a permanent magnet. Unbound species were washed off with the buffer. Following that, the external magnetic field was removed. Magnetically captured proteins were collected and subjected to gel filtration.

### (3) Final Polishing Purification Stage—Gel Filtration

This fractionation stage was used to isolate only one component, namely separate monomers from aggregates and higher oligomers, on the basis of differences in their sizes.

As in ion exchange chromatography, preparative work, included choice of the gel media and the running buffer. “Superdex×200 pg” was selected as a column medium, which is based on highly cross-linked porous aragose particles to which dextran has been covalently bonded. This medium is designed to provide excellent gel filtration characteristics, combined with high chemical and physical stability and covers a broad fractionation range for globular proteins (1×10⁴-10⁶ Daltons).

Fractionation is achieved due to the fact that species with higher molecular weights will be filtered through the column faster. The mobile phase (running buffer) was 0.2 M Na acetate pH=5, at a flow rate 1 ml/min. The column was calibrated with apoferritin, with a UVNis detection system in place.

Following gel filtration, a monomer fraction of magnetoferritin was isolated and used for crystallization experiment.

### EXAMPLE 3

### Crystallization of Magnetoferritin

The batch crystallization method was used in all experiments (50 μl volume). For incubation the following reservoirs were used, all maintained at 20° C.:
(i) 24 well plates that were sealed with transparent tape;
(ii) quasi- two dimensional sealed glass chambers, constructed from two glass cover slips with a spacer in between them (−1 mm); and
(iii) plastic sample tubes (volume 0.2 ml).

Reservoirs “i” were used to prepare samples for observations on the optical microscope with 50× magnification; reservoirs “ii” were used for Raman spectroscopy and subsequent optical imaging; and reservoirs “iii” were used for magnetic characterization by vibrating sample magnetometer (VSM) and SQUID.

All crystallization samples contained 0.2 M sodium acetate buffer to maintain pH=5, cadmium sulphate at 3.2% w/w as crystallizing agent, and protein solution at concentrations >1 mg/ml. To achieve supersaturation equal amounts (1:1) CdSO₄ 3.2% w/w in 0.2 M sodium acetate buffer and magnetoferritin at concentrations >1 mg/ml were mixed.

Control experiments were run in parallel using apoferritin.

### RESULTS

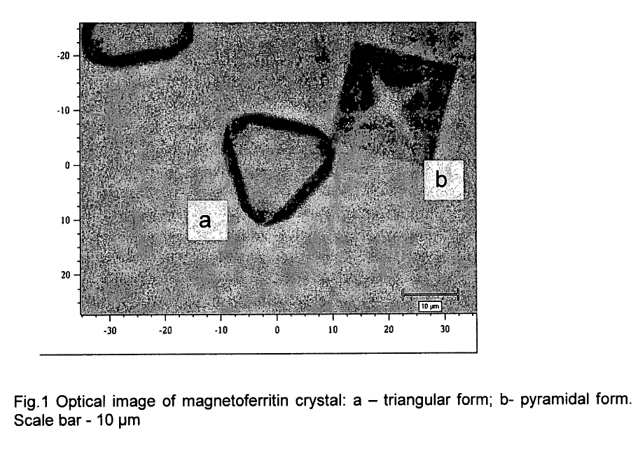

It is known that ferritin molecules crystallize in octahedral crystals with {111} in the crystal habit. Magnetoferritin crystals with three types of orientations were obesrved: {111} plane parallel to the substrate (triangle form—FIG. 1, a), {110} parallel to the substrate and {100} parallel to the substrate (like a pyramid, FIG. 1, b). We also observed some dendrimer formation.

Raman spectroscopy confirmed that the observed crystals are proteins, and VSM measurements show they are magnetic.

Example 3 shows that the ferritin protein will still crystallise following chemical modification to its core.

1-48. (canceled)

49. A 3D functional protein crystal, wherein each protein in the 3D functional protein crystal comprises a cavity containing a core nano-particle, the core nano-particle comprising an elemental metal, a metal alloy or a metal compound, with the proviso that the protein is not apoferritin, Dpr or E. Coli dps when the core nano-particle is ferrihydrite.

50. The 3D functional protein crystal of claim 49, selected from a ferritin, a capsid, a virus, a bacteriophage, a lysozyme, a flagellar LP ring, a microtubule and a chaperonin.

51. The 3D functional protein crystal of claim 50, which is a human apoferritin or a horse apoferritin.

52. The 3D functional protein crystal of claim 49 or 50, wherein each protein in the 3D functional protein crystal is a recombinant protein.

53. The 3D functional protein crystal of claim 49, wherein the core nano-particle comprises:
an elemental metal selected from the group consisting of iron, cobalt, nickel, copper, palladium, platinum, silver and gold; or
an alloy comprising at least one of aluminium, barium, bismuth, cerium, chromium, cobalt, copper, dysprosium, erbium, europium, gadolinium, gold, holmium, iron, lanthanum, lutetium, manganese, molybdenum, neodymium, nickel, niobium, palladium, platinum, praseodymium, promethium, samarium, silver, strontium, terbium, thulium, titanium, vanadium, ytterbium, and yttrium; or
a semiconductor comprising at least one of gold sulphide, gold selenide, gold telluride, cadmium sulphide, cadmium selenide, cadmium telluride, zinc sulphide, zinc selenide, zinc telluride; or
a ferri- or ferro-magnetisable elemental metal or metal alloy; magnetite; maghemite; a compound of formula MeFe₂O₄, where Me is a divalent transition metal selected from Ti, Cr, Mn, Co, Ni, and Cu; or a ferri- or ferro-magnetisable transition metal sulphide.

54. The 3D functional protein crystal of claim 49, wherein the core nano-particle has a largest dimension in the range from 1 to about 100 nm.

55. The 3D functional protein crystal of claim 49, wherein each dimension of the crystal, when measured through the center of mass, is greater than 125 nm.

56. The 3D functional protein crystal of claim 49, wherein each dimension of the crystal, when measured through the center of mass, is greater than 1.25 82 m.

57. The 3D functional protein crystal of claim 49, wherein each dimension of the crystal, when measured through the center of mass, is greater than 125 μm.

58. A method of synthesizing a 3D functional protein crystal, the method comprising:
(i) preparing a plurality of a protein in a solution, wherein the protein comprises a cavity capable of accommodating a core nanoparticle;
(ii) synthesizing the core nanoparticle within the cavity of at least some of the plurality of the protein in the solution; the core nano-particle comprising an elemental metal, a metal alloy or a metal compound;
(iii) separating a plurality of monomeric proteins, each comprising the core nano-particle, from the solution; and
(iv) subjecting the plurality of monomeric proteins to crystallization conditions to form the 3D functional protein crystal;
with the proviso that the protein is not apoferritin, Dpr or E. Coli dps when the core nano-particle is ferrihydrite.

59. The method of claim 58, wherein the protein is selected from a ferritin, a capsid, a virus, a bacteriophage, a lysozyme, a flagellar LP ring, a microtubule and a chaperonin.

60. The method of claim 58, wherein the protein is human apoferritin or horse apoferritin.

61. The method of claim 59 or 60, wherein the protein is a recombinant protein.

62. The method of claim 59, wherein the core nanoparticle is prepared by a process in which the solution of the protein is combined with a source of ions of the appropriate metal, the metal alloy or the metal compound of which the core nanoparticle is comprised.

63. The method of claim 62, wherein the source of ions is added incrementally to the solution of the protein to provide more than 1 ion of the appropriate metal, the metal alloy or the metal compound per protein per iteration, and fewer than 200 ions of the appropriate metal, the metal alloy or the metal compound per protein per iteration.

64. The method of claim 62 or 63, wherein separating monomeric proteins, each comprising the core nano-particle, from the solution is achieved by sequentially subjecting the solution containing the protein to an ion exchange chromatography, followed by a gel filtration.

65. The method of claim 64, wherein the core nanoparticle exhibits paramagnetism, superparamagnetism, ferri- or ferro-magnetism.

66. The method of claim 65, wherein the product of the ion exchange chromatography is subjected to a magnetic fractionation prior to the gel filtration, optionally wherein the magnetic fractionation comprises High Gradient Magnetic Separation.

67. The method of claim 64, wherein the ion exchange chromatography comprises a step salt gradient elution.

68. The method of claim 58, wherein subjecting the plurality of monomeric proteins to crystallization conditions to form the 3D functional protein crystal comprises a batch crystallization wherein the plurality of monomeric proteins is combined with a buffer and a crystallization agent and then allowed to crystallize.

69. The method of claim 68, wherein the buffer is selected to maintain the pH of the solution at a value within about 3 units of the isoelectric point of the protein and the crystallization agent is sodium chloride, cadmium sulphate or PEG.

70. A device comprising the 3D functional protein crystal of claim 49, wherein the device is selected from a drug and/or thermotherapy delivery system, a magnetic data storage medium, an optical waveguide, a sub-diffraction limited lens, an enhancement agent for Raman spectroscopy, a photonic band gap device, a biocompatible implant, and a surface Plasmon resonance biosensor.

71. A method of using of the 3D functional protein crystal of claim 49, wherein the use comprises integration of the 3D functional protein crystal into a data storage medium, an optical waveguide, a sub-diffraction limited lens, an enhancement agent for Raman spectroscopy, a photonic band gap device, a biocompatible implant, a surface Plasmon resonance biosensor or a drug and/or thermotherapy delivery system.
